(19) United States
(12) Patent Application Publication
(10) Pub. No.: US 2007/0237839 A1
(43) Pub. Date: Oct. 11, 2007

(54) Compositions and methods for uterine relaxing

(75) Inventors: Chia-Li Wei, Keelung City (TW); Meng-Hwan Lee, Yunlin County (TW); Chun-Ying Lin, Taipei County (TW); Jay Hua, Hsinchu City (TW); Ying-Chieh Tsai, Taipei City (TW)

Correspondence Address: BIRCH STEWART KOLASCH & BIRCH, PO BOX 747, FALLS CHURCH, VA, 22040-0747, US

(73) Assignee: Yangsen Biotechnology Co., Ltd.

(21) Appl. No.: 11/392,698

(22) Filed: Mar. 30, 2006

(51) Int. Cl. A61K 36/53

(52) U.S. Cl. 424/725; 424/745

(57) ABSTRACT

A method for treating/relief of women's menstruation pain. The pain is reduced or relieved by externally administering an effective amount of an essential oil obtained from origanum, anthopogan, perrila, herba moslae, elemi, cuban oregano and/or a combination thereof.

### BACKGROUND OF THE INVENTION

1. Field of the Invention

The present invention relates to pharmaceutical compositions, and in particular relates to pharmaceutical compositions and methods for relieving menstruation pain.

2. Description of the Related Art

It is common for women to experience painful cramps when they have their monthly period (menstruation), especially at their young age. Some women also get some pain or a feeling of congestion in the abdomen in the days before their period. Every month while women are in their reproductive years, the uterus builds up the endometrium (inner lining of the uterus) with blood and other fluids. The endometrium is intended to provide the nourishment that an embryo would need if the woman were to become pregnant. If, after ovulation (release of an egg into the uterus or womb), the woman does not become pregnant, then the uterus sheds the endometrium over a few days (menstruation or period).

Therefore, the muscles in the uterus contract and then relax, to squeeze the endometrium, break it down and get it moving out of the uterus. The fluid leaves the uterus through the cervix, and then flows out of the woman's body through the vagina. Most contractions of the muscles in the uterus are not felt. Sometimes, though, contractions are strong and frequent, causing pain. These are often called menstrual cramps. Period pain can be felt just in the abdomen, or it can spread up and around the back, and down the legs. Some women also experience nausea, vomiting or even diarrhoea.

The medical term for painful periods is dysmenorrhea. Most women (as many as 70% of women) will get some amount of pain when they have a period. For about one in ten of these women, that pain can be so bad that for one to three days each month they are unable to carry on with their lives normally. Period pain, as well as severe period pain, is even more common in teenage women, affecting as many as 70% to 90% of teenagers. After a teenage woman has been menstruating for a few years, the pain is likely to at least reduce, and will stop for some. Many women will also have less period pain after they have a baby.

Medically, there are two categories of period pain: primary and secondary. Primary period pain is when it is only the menstrual cramps causing the pain. Secondary period pain is caused by a disease or condition (such as cysts on the ovaries, or endometriosis—where endometrium grows in other places than the uterus). If period pain is severe or getting worse, it needs to be investigated by a doctor to evaluate the possible cause.

In addition to taking over-the-counter analgesic, one treatment for period pain is to regulate or reduce levels of prostaglandin (PG) by taking either the oral contraceptive pill (the pill), or NSAIDs (non-steroidal anti-inflammatory drugs). PGs will cause the uterine muscles to contract to get the endometrium moving out of the uterus and the contractions (cramps) may cause pain.

In spite of their effectiveness, the presence of side effects from these drugs to liver, kidneys and digestive system limit their clinical use. A feasible alternative of natural herb therapy needs therefore to be considered.

### BRIEF SUMMARY OF THE INVENTION

An object of the invention is to provide period pain-relieving essential oils for women when it is applied in an amount by which sufficient uterine relaxing effect can be obtained.

In an embodiment, the present invention provides a uterine relaxing composition with a sufficient amount of an essential oil extracted from origanum, anthopogan, perrila, herba moslae, elemi, cuban oregano or a combination thereof, which can be used externally to reduce or relieve women's menstruation pain and dysmenorrhea.

In another embodiment, the uterine relaxing composition contains carvacrol, 1-β-pinene, and/or limonene, which may further include an essential oil of origanum, anthopogan, perrila, herba moslae, elemi, cuban oregano or a combination thereof, to improve the uterine relaxing activity.

A detailed description is given in the following embodiments and experiment results.

### BRIEF DESCRIPTION OF THE DRAWINGS

The present invention can be more fully understood by reading the subsequent detailed description and examples with references made to the accompanying drawings wherein:

FIG. 1 shows the inhibitory effects of essential oils from origanum, anthopogan, perrila, herba moslae, elemi and cuban oregano on oxytocin- (OTC-), PGF_{2α}- and KCl-stimulated rat uterine muscle contraction; and

FIG. 2 shows the inhibitory effects of carvacrol, 1-β-pinene, limonene, thymol and linalool on OTC-, PGF_{2α}- and KCl-stimulated rat uterine muscle contraction.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined bye reference to the appended claims.

Essential oils, also called volatile or ethereal oils, are aromatic oily liquids obtained by expression, fermentation, enfleurage or extraction from plant material such as flowers, buds, seeds, leaves, twigs, bark, herbs, wood, fruits and roots, referring to “Essential oils: their antibacterial properties and potential applications in foods—a review” (Sara Burt, International Journal of Food Microbiology 2004; 94: 223-53). Essential oils are usually composed of tiny molecules, this allows them to penetrate the skin easily and work into the body during externally topical use.

According to the invention, it is found that six essential oils obtained from origanum, anthopogan, perrila, herba moslae, elemi, and cuban oregano are effective for uterine relaxing, which will be helpful for period pain relieving by external use.

Experiment Results of Uterine Relaxing Activity

TABLE 1
Materials-Essential oil

| Essential oil sample | Common name | Scientific name |
|---|---|---|
| A | origanum | Origanum vulgare |
| B | anthopogan | Rhododendron anthopogan |
| C | perrila | Perilla frutescens |
| D | herba moslae | Mosla chinensis |
| E | elemi | Canarium luzonicum |
| F | cuban oregano | Plectranthus amboinicus |

The essential oils of origanum, anthopogan, perrila, herba moslae and elemi were purchased from Heng Yi Trading Co., Ltd. (Taiwan), Natural Resources Industries (P) Ltd. (Nepal), Jiangxi Jishui TongRen Natural & pharmaceutical oil factory (China), HuaLong Magnolia Development Co., Ltd. (China) and A. M. Aromatic & Essential Oils (India), respectively. Essential oil of cuban oregano was extracted by hydrodistillation of leaf of the plant from Taiwan using a Clevenger-type apparatus.

Uterine Relaxing Activity

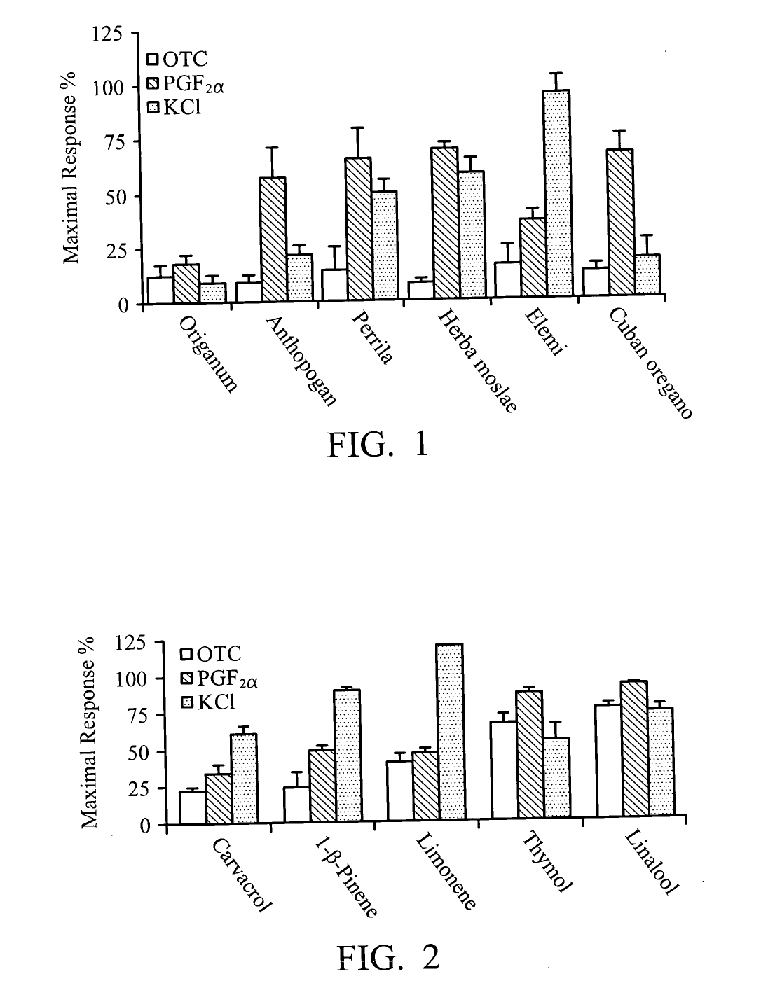

Uterine relaxing activities on OTC- and KCl-stimulated rat uterine muscle contraction were carried out according to the method described by Ostad S N, Soodi M, Shariffzadeh M, Khorshidi N, and Marzban H. (The effect of fennel essential oil on uterine contraction as a model for dysmenorrhea, pharmacology and toxicology study. Journal of Ethnopharmacology 2001; 76: 299-304.). Female virgin Sprague-Dawley rats (200 g to 300 g) were obtained from the Laboratory Animal Center at Yang-Ming University. They were primed with 5 mg/kg of estradiol (Sigma-Aldrich) 24 hours before the experiments, and then sacrificed by CO₂ asphyxiation. Uterine strips free from adhering tissues were mounted under a resting tension of 1 g in a 10 ml organ bath containing dejalon solution gassed with carbogen (95% O₂ and 5% CO₂) at 31° C., which were then allowed to equilibrate for at least 1 hour. To evaluate the effect on the contractile response induced by PGF_{2α}, a modified Locke-Ringer solution and a maintained temperature of 28° C. were used according to the method described by Murata P, Hayakawa T, Satoh K, Kase Y, Ishige A, and Sasaki H. (Effects of Dai-kenchu-to; a herbal medicine, on uterine and intestinal motility. Phytotherapy Research 2001; 15: 302-6). Contractions were recorded by force displacements transducers (Grass Instruments USA) using MP100 workstation software (Biopac Systems USA) on a PC. The results of inhibitory activities of the essential oils are shown in FIG. 1.

FIG. 1 shows the comparison of the inhibitory effects of essential oils from origanum, anthopogan, perrila, herba moslae, elemi and cuban oregano on OTC-, PGF_{2α}-, and KCl-stimulated rat uterine muscle contraction. The muscle strip was pretreated with 80 μg/ml of essential oils for 10 min before OTC (10 mU/ml), PGF_{2α} (1 μM) or KCl (60 mM) treatment. The maximal response percentage represents the muscle contraction degree after treatment, comparing with no essential oil pretreatment as the control group (100%). Data were presented as mean±standard error of three independent experiments. As shown in FIG. 1, for OTC-stimulated rat uterine muscle contraction, the muscle contraction responses for all six essential oils are 8% to 16%, which indicates that these essential oils is effective for inhibiting 84% to 92% uterine muscle contraction than the control group (i.e. no essential oil treatment). Similarly, for PGF_{2α}-stimulated rat uterine muscle contraction, origanum and elemi show the strongest inhibition (82% and 66%, respectively) of muscle contraction, and anthopogan, perrila, herba moslae, and cuban oregano show moderate inhibition (31% to 43%) as well. For KCl-stimulated rat uterine muscle contraction, origanum, anthopogan, and cuban oregano show 78% to 91% inhibition. Perrila and herba moslae also show moderate inhibition (50% and 42%, respectively) of muscle contraction.

Dysmenorrhea Model in Mice

Another dysmenorrhea model in mice by Yang et al. (Yang M.-H., Jin Z.-H., Guo Y.-F., Zhu P.-Q., Chen W.-J. Study on regulation action of Tiaojinghuoxue capsules on menstruation. Chinese Traditional Patent Medicine 2000; 22: 717-9.) with modifications was also conducted to evaluate the activity of the essential oils. Thirty-five female ICR mice (30 g to 40 g) from the Laboratory Animal Center at National Taiwan University College of Medicine were grouped into three. One ml/kg of jojoba oil, origanum or anthopogan essential oils (30% in jojoba oil, respectively) were rubbed on belly of mice once a day during days 1-5 and 8-12, followed by intraperitoneal injection of diethylstilbestrol (10 mg/kg) once a day during days 9-11. After 30 min of oil rubbing on day 12, OTC (15 U/kg) was intraperitoneally injected and the induced twisting numbers in 30 min were recorded. The experiment results are shown in TABLE 2.

TABLE 2
Effects of essential oils from origanum and anthopogan
on dysmenorrhea mouse

| | Number of | Twisting | Twisting |
|---|---|---|---|
| Groups | twisting mouse | number^{a} | percent |
| jojoba | 12 | 21.8 ± 8.1 | 100 |
| origanum | 12 | 5.1 ± 5.3 | 23 |
| anthopogan | 12 | 5.9 ± 4.4 | 27 |

^{a}Mean ± standard error was shown.

A similar dysmenorrhea model experiment was also conducted for herba moslae and perrila essential oils and the experiment results are shown in TABLE 3.

TABLE 3
Effects of essential oils from herba moslae
and perrila on dysmenorrhea mouse

| | Number of | Twisting | Twisting |
|---|---|---|---|
| Groups | twisting mouse | number^{a} | percent |
| jojoba | 12 | 14.5 ± 6.2 | 100 |
| herba moslae | 12 | 6.9 ± 4.2 | 48 |
| perrila | 12 | 3.8 ± 3.5 | 26 |

^{a}Mean ± standard error was shown.

According to the results shown in TABLE 2 both origanum and anthopogan essential oils could decrease the twisting times (77% and 73%) of dysmenorrhea mice induced by OTC, comparing to the control group (jojoba oil). According to the results shown in TABLE 3, both perrila and herba moslae oils could also decrease the twisting times (74% and 52%) of dysmenorrhea mice.

Chemical Analysis of the Essential Oils

The content of the six essential oils were further analyzed by gas chromatography-mass spectrometry (GC-MS). The chemical composition of each essential oil was determined using an Agilent 6890N gas chromatography., equipped with a HP-5MS capillary column (30 m×0.25 mm; film thickness; 0.25 μm) and an Agilent 5973 mass spectrometer as detector. Injector and detector (MS transfer line) temperatures were set at 250° C. and 285° C., respectively. The carrier gas was helium (1 ml/min), and 1 μl of diluted sample (1/10 in isopropanol) was injected by an autosampler (Agilent 7683 Series). The oven temperature was programmed as follows: 40° C. (5 min), 40° C. to 180° C. (3° C. min⁻¹), 180° C. to 200° C. (6° C. min⁻¹), 200° C. to 250° C. (8° C. min⁻¹), and 250° C. (3 min). The oil components were identified by comparison of their retention indices and mass spectra with the GC-MS computer database (Wiley 275.L). Parts of the content results of the six essential oils are shown in TABLE 4.

TABLE 4
Content percentage of compounds identified
by GC-MS from the essential oils

| | Content % of compounds identified by GC-MS | | | | | |
|---|---|---|---|---|---|---|
| | from the essential oils | | | | | |
| Com- | | | | herba | | cuban |
| pound | origanum | anthopogan | perrila | moslae | elemi | oregano |
| carvacrol | 35.66 | — | — | 6.39 | — | 57.98 |
| 1-β-pinene | — | 6.92 | — | — | — | — |
| limonene | — | 8.77 | 20.32 | 31.36 | 29.28 | 1.24 |
| thymol | 30 | — | — | 3.54 | — | — |
| linalool | 6.05 | — | 1.38 | — | — | 0.20 |

A comparison of the inhibitory effects of carvacrol, 1-β-pinene, limonene, thymol and linalool on OTC-, PGF_{2α}- and KCl-stimulated rat uterine muscle contraction was conducted. The muscle strip was pretreated with 20 μg/ml of compounds for 10 min before OTC (10 mU/ml), PGF_{2α} (1 μM) or KCl (60 mM) treatment. The maximal response percentage represents the muscle contraction degree after treatment, comparing with no essential oil pretreatment as the control group (100%). Data were presented as mean±standard error of three independent experiments. The results are shown in FIG. 2.

As shown in FIG. 2, for OTC-stimulated rat uterine muscle contraction, carvacrol, 1-β-pinene, limonene, thymol and linalool all relieve the muscle contraction responses (24% to 78%). For PGF_{2α}-stimulated rat uterine muscle contraction, carvacrol, 1-β-pinene and limonene show 51% to 66% inhibition of muscle contraction, whereas thymol and linalool didn't show obvious inhibition. For KCl-stimulated rat uterine muscle contraction, carvacrol, thymol and linalool show 27% to 46% of inhibition.

According to the above experiment results, it is shown that all six essential oils from origanum, anthopogan, perrila, herba moslae, elemi, and cuban oregano exhibit relaxing activity of uterine muscle contraction in different degrees and thereby the six essential oils can be used solely or in combination as a uterine relaxing composition for treating period pain or dysmenorrhea. Moreover, the compounds, carvacrol, 1-β-pinene, limonene, thymol and linalool, isolated from the six essential oils, also show relaxing activity of uterine muscle contraction, and thereby, the compounds can also be used as a uterine relaxing composition for treating period pain or dysmenorrhea.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A uterine relaxing composition, comprising: a sufficient amount of an essential oil extracted from Origanum vulgare (origanum), Rhododendron anthopogan (anthopogan), Perilla frutescens (perrila), Mosla chinensis (herba moslae), Canarium luzonicum (elemi), Plectranthus amboinicus (cuban oregano) and/or a combination thereof.

2. The uterine relaxing composition as claimed in claim 1, wherein the essential oil comprises carvacrol.

3. The uterine relaxing composition as claimed in claim 1, wherein the essential oil comprises 1-β-pinene.

4. The uterine relaxing composition as claimed in claim 1, wherein the essential oil comprises limonene.

5. The uterine relaxing composition as claimed in claim 1, wherein the essential oil comprises thymol.

6. The uterine relaxing composition as claimed in claim 1, wherein the essential oil comprises linalool.

7. A method for treating/relieving menstruation pain by administering an effective amount of a uterine relaxing composition comprising a sufficient amount of an essential oil extracted from Origanum vulgare (origanum), Rhododendron anthopogan (anthopogan), Perilla frutescens (perrila), Mosla chinensis (herba moslae), Canarium luzonicum (elemi), Plectranthus amboinicus (cuban oregano) and/or a combination thereof.

8. A uterine relaxing composition, comprising a sufficient amount of carvacrol, 1-β-pinene, limonene and/or a combination thereof.

9. The uterine relaxing composition as claimed in claim 8, comprising an essential oil of Origanum vulgare (origanum).

10. The uterine relaxing composition as claimed in claim 8, comprising an essential oil of Rhododendron anthopogan (anthopogan).

11. The uterine relaxing composition as claimed in claim 8, comprising an essential oil of Perilla frutescens (perrila).

12. The uterine relaxing composition as claimed in claim 8, comprising an essential oil of Mosla chinensis (herba moslae).

13. The uterine relaxing composition as claimed in claim 8, comprising an essential oil of Canarium luzonicum (elemi).

14. The uterine relaxing composition as claimed in claim 8, comprising an essential oil of Plectranthus amboinicus (cuban oregano).

15. The uterine relaxing composition as claimed in claim 8, comprising an essential oil of Origanum vulgare (origanum), Rhododendron anthopogan (anthopogan), Perilla frutescens (perrila), Mosla chinensis (herba moslae), Canarium luzonicum (elemi), Plectranthus amboinicus (cuban oregano) and/or a combination thereof.

16. A method for treating/relieving menstruation pain by administering an effective amount of the uterine relaxing composition as claimed in claim 15.
